(19) United States
(12) Patent Application Publication
(10) Pub. No.: US 2026/0363571 A1
(43) Pub. Date: Nov. 19, 2026

(54) Support for Universal Communication with Active Implantable Medical Devices in Emergency Support Use Cases

(71) Applicant: BIOTRONIK SE & Co. KG, Berlin (DE)

(72) Inventors: Dirk MUESSIG, West Linn, OR (US); R. Hollis WHITTINGTON, Portland, OR (US); Patrick L. PARISH, Portland, OR (US); Brian M. TAFF, Portland, OR (US)

(73) Assignee: BIOTRONIK SE & Co. KG, Berlin (DE)

(21) Appl. No.: 19/153,799

(22) PCT Filed: Jan. 26, 2024
(86) PCT No.: PCT/EP2024/051914

§ 371 Date: Aug. 5, 2025

Related U.S. Application Data
(60) Provisional application No. 63/445,116, filed on Feb. 13, 2023.

(30) Foreign Application Priority Data
Mar. 15, 2023 (EP) 23161978.4

(51) Int. Cl. A61B 5/07; A61B 5/00; A61N 1/372

(52) U.S. Cl. CPC A61B 5/076; A61B 5/0031; A61B 5/686; A61N 1/37288; A61B 2505/01; A61B 2562/08

(57) ABSTRACT

An implant for monitoring of a physiological parameter associated with a health state of a patient is configured to receive a trigger requesting a predetermined discovery signal and to transmit the predetermined discovery signal.

### CROSS-REFERENCE TO RELATED APPLICATIONS

This application is the United States National Phase under 35 U.S.C. § 371 of PCT International Patent Application No. PCT/EP2024/051914, filed on Jan. 26, 2024, which claims the benefit of European Patent Application No. 23161978.4, filed on Mar. 15, 2023, and U.S. Provisional Patent Application No. 63/445,116, filed on Feb. 13, 2023, the disclosures of which are hereby incorporated by reference herein in their entireties.

### TECHNICAL FIELD

The present invention relates to an implant, a medical device, as well as a respective system, method, and computer program for medical implant detection.

### BACKGROUND

It is a common situation that a patient arrives in a clinical emergency department in need of medical care in conditions where they are unable to provide meaningful guidance to the attending staff regarding the nature and type of their in-body active implantable medical devices/implants. Lacking verbal guidance and insights from the patient, health professionals are, in turn, often left needing to search the patient's personal belongings for carried device identification cards and/or attempt to access medical records to gain such insights. Establishing communication and performing subsequent implant interrogations often involves time-consuming procedures that can include fluoroscopic imaging of the patient in search of a device manufacturer visual identifier and/or trial and error procedures using a potential multitude of implant-and/or manufacturer-specific programmers from a variety of medical product manufacturers.

Therefore, there is still a need to further improve the methods and devices for in-body active implantable medical device identification.

The above need is at least partly met by the aspects as described herein.

The present disclosure is directed toward overcoming one or more of the above-mentioned problems, though not necessarily limited to embodiments that do.

### SUMMARY

According to one aspect of the invention, an implant for monitoring of a physiological parameter associated with a health state of a patient is provided. The implant comprises means for receiving a trigger requesting a predetermined discovery signal and means for transmitting the predetermined discovery signal.

Generally speaking, the trigger may be seen as an anonymous “Who is there?”-message which is not specifically directed at any device but may be received by any plurality of devices, e.g., implants, and the discovery signal provides an “I am here!”-message to any device capable of receiving the discovery signal. The discovery signal may therefore provide an opportunity to identify the presence of an implant in a patient's body, the device type and manufacturer quickly and efficiently. This may significantly reduce the time needed to identify an implant by providing a universal identification scheme. Said reduction of time may sideline many of the traditional needs for searching patient records, subjecting the patient to fluoroscopic imaging, and/or enforcing trial and error procedures using a potential multitude of manufacturer-specific programmers. The invention may be especially beneficial for patients in incapacitated conditions but may also offer benefits in cases where a conscious patient might not be aware of their specific implant type.

In other examples, the implant may transmit a discovery signal as described herein without requiring receiving a trigger. It may, e.g., transmit a discovery signal periodically and/or when a certain event occurs.

The monitored physiological parameter may further, e.g., be determined by the implant and recorded by it, e.g., in an implant log file.

The implant may, e.g., be any at least partly implantable medical device, for example a neurostimulation device, a pacemaker, an electrocardiography device, etc. The implant may, for example, be any device capable of monitoring at least one physiological parameter of the respective patient (e.g., a sensor) and/or to deliver a therapy (e.g., a neurostimulation device). The implant may comprise separate means configured to receive the trigger and to transmit the discovery signal or have one combined means for receiving the trigger and transmitting the discovery signal. The means for transmitting and receiving may be based for example on wireless communication methods like, e.g., Bluetooth low energy.

The trigger requesting a predetermined discovery signal to be transmitted by any device receiving the trigger may be any signaling which can evoke the transmission of said discovery signal by, e.g., a receiving implant.

Such trigger may only evoke the transmission of a discovery signal when the trigger is received by an according implant. Therefore, the transmission of said discovery signal evidently indicates the presence of the implant transmitting the discovery signal and the discovery signal serves the purpose of allowing the implant to be discovered and/or to be identified.

The exchange of triggers and discovery signals as described herein may for example occur without demanding that the triggering device and responding device are paired within a devoted/prescribed communication link or otherwise engaged using any direct physical connection.

In an exemplary embodiment, the trigger does not comprise an instruction determining the content of the requested discovery signal and/or the content of the discovery signal may be predetermined before receiving the trigger.

In an example, the trigger does not comprise an instruction determining the content of the discovery signal but merely requests that a discovery signal is being sent by any implant receiving the trigger combined with the discovery signal being predetermined before receiving the trigger may in combination increase safety and reliability of sensitive patient data communication. Additionally, or alternatively, the discovery signal may comprise at least an implant identification and/or a patient identification, preferably in an encrypted manner. The patient identification information may be collected by the medical device (for scanning) subject to added measures such as subsequent placement of the medical device (for scanning) in direct contact with the patient.

The discovery signal may comprise at least an implant identification and a patient identification provides the opportunity to any receiver of a plurality of such discovery signals to differentiate between the implants of different patients and have an overview on which patient has which implants, e.g., in situations when patients share a room, etc.

The patient identification may, e.g., comprise a patient name, a patient birth date, a patient number, a patient emergency contact (e.g., of a relative or a general practitioner), a patient email address, a patient telephone number, health insurance information, information to access such information in an electronic patient file of an electronic health record/data base, etc.

The device identification may, e.g., comprise a device type (number and/or name) and/or a manufacturer identification (number and/or name). For example, device type and manufacturer may be combined in a common identification (number and/or name). The device identification may, e.g., comprise the patient identification in form of a (decoded) patient identification number which may further be used to access additional patient information stored in an electronic health record/data base. This may also be helpful in situations when not only the implants but also the patient themselves are unknown to the attending health professional but may be identified by entering the patient identification number into the electronic health record and/or by directly reading out the patient's name from the discovery signal. This may improve treatment success chances as more potentially decisive information on the patient may be accessed.

The discovery signal may comprise further information, e.g., a signal strength indication relating to the intensity of the discovery signal transmitted by the implant, a location information on the position where the implant is positioned within the patient's body, emergency contact information of health professionals involved in previous treatments of the patient, etc.

For example, this communication protocol may function without establishing and sustaining a connection in between the transmitter of the trigger and the implant receiving the trigger which may save energy and increase safety further.

The trigger may for example simply comprise a device and manufacturer independent request to send a discovery signal of any form, as the form and content of the discovery signal is being determined by the implant and not by the transmitter of the trigger.

The discovery signal is predetermined in such that its form and content are independent of the received trigger. In the underlying mechanism receiving the trigger directly evokes the transmission of the predetermined discovery signal. Said discovery signal maybe stored in full or in parts, and, e.g., assembled prior to sending the discovery signal, within the implant and may be sent in that form in in response to receiving the trigger. Accordingly, neither the form nor the content of the discovery signal is altered based on the received trigger.

In an exemplary embodiment the discovery signal does not include any patient identification and instead demands near proximity of an interrogating device (i.e., by resting something in contact with the patient's chest) to simply collect all the device types implanted within the patient of interest. This could avoid any need for encryption of patient information and avoids the confusion on which identified devices reside in which patient.

In an exemplary embodiment of the implant, the trigger may be undirected (e.g. a broadcast or a general petition) and/or may comprise no implant-specific information. For example, the trigger may not be directed to a specific implant but rather constitute a broadcast to reach all available implants close by.

The capability of the implant to receive and be triggered by a non-implant-specific trigger may be advantageous for the following reason. It allows an open and unbiased scan for implants in the patient which may even be compatible with a growing plurality of (new) implants on the market: E.g., implants that are developed after the transmitter of the trigger may be unknown to exist to the transmitter of the trigger but may still be addressed by the trigger.

The trigger signal comprising no implant-specific information relates to a general trigger comprising an anonymous request, instructing any device receiving the trigger to send a discovery signal to be then detected by the transmitter of the trigger or a second associated device.

In an example, the implant may further be configured to operate in a default receiving state, to transition into a discovery state upon receiving the trigger for a predetermined period, wherein the discovery signal is transmitted in the discovery state, and to transition into the default receiving state after the predetermined time period has lapsed.

This workflow of the implant optimizes the operation state in which the implant is at any time such that, e.g., energy consumption is minimized, for example by sending the discovery signal only for a limited time after receiving the trigger. This may therefore also be well-adjusted to the workflow of any device capable of receiving the discovery signal as this may be perceptive after the trigger is being sent. Not sending the discovery signal at any time but only when a suitable trigger evokes it, and thus indicates an authorized request in form of a suitable trigger, prevents or reduces the risk for unwanted implant discovery and/or identification by unauthorized parties.

When in the default receiving state, the implant may not transmit any signals to its surrounding which facilitates both an energy-saving and safe mode wherein the implant may not be detected easily by unauthorized devices. In the default receiving state, it may, however, be perceptive to incoming triggers at any time or during periodically repeating time intervals. In an example, the default receiving state may comprise active phases with a duration T_{A} and inactive phases with a duration T_{I}, which alternate periodically and may optionally be modulated by a quasi-random time delay T_{D}. While in the active phase, the implant may receive incoming triggers, it is not perceptive for incoming triggers in the inactive phase and may save energy in that time. Any trigger transmitter may, however, send multiple triggers in succession such that at least one trigger may coincide with an active phase of the implant to evoke a discovery signal. The quasi-random modulation may increase the likelihood for this.

The implant may for example be further configured to transmit a plurality of discovery signals, wherein each discovery signal preferably has a different content and/or different properties. These properties may, e.g., relate to signal strength, size, encryption, delay duration, etc. Additionally, or alternatively the implant may be configured to update the content of the discovery signal, preferably based at least in part on physiological data acquired by the implant.

Providing a plurality discovery signals may increase the amount of information that may be communicated through the discovery signals and provides the opportunity to transmit data independently from each other when, e.g., they are not wanted to be sent together for data safety reasons.

For example, receiving a trigger may cause the implant to transmit two or more discovery signals one after another, e.g., separated by equal time intervals. For example, a first discovery signal may comprise the device identification and the patient identification, a second discovery signal may comprise the device identification and the manufacturer identification, and a third discovery signal may comprise the device identification and information on the position of the implant within the patient's body and/or physiological data of the patient. These or further discovery signals may further comprise any information described herein. Alternatively, e.g., at least two discovery signals may comprise the same and/or similar content.

Further, a plurality of triggers and/or discovery signals may be received/transmitted that are nominally identical in terms of their payload content, except that they simply serve to enforce different delay durations between the incoming trigger and the responding discovery signals. This type of approach is particularly helpful when multiple implants are “in range” or close to each other, and it should be avoided that they respond at the same time (and thus colliding/interfering).

The physiological data of the patient optionally provided by the implant may be acquired by the implant or entered, e.g., manually by a health professional via an implant-specific programmer. The physiological data may, e.g., comprise any physiological data the implant is capable of monitoring and/or processed versions thereof. These physiological data may therefore for example be implant dependent. In the example of an electrocardiography (ECG) implant, the physiological data may, e.g., comprise parameters extracted from an ECG: An ECG waveform typically comprises three characteristic components: a so-called P wave, which represents depolarization of the atria, a subsequent QRS complex, which represents depolarization of the ventricles, and finally a T wave, which represents repolarization of the ventricles. The extracted parameter(s) may thus for example comprise a QT-time, a PQ-time, a QRS-width, a variability of the heart rate, and/or data on whether, how often, and/or when bradycardia and breaks, atrial and/or ventricular extrasystoles (e.g., two subsequent ventricular extrasystoles (a couplet) or three subsequent ventricular extrasystoles (a burst)), high ventricular rates, breaks, cardiac inotropy, and/or tachycardia (e.g., atrial fibrillation/flutter with pre-excitation, atrial tachycardia, ventricular tachycardia, superventricular tachycardia, and/or torsades de pointes) occur. The physiological data and/or any related information may, e.g., be recorded periodically or event-triggered. For example, the implant may save the time of the last time a parameter was recorded that may indicate a health risk of the patient like, e.g., an increased blood pressure.

In another example, the physiological data may alternatively or additionally be provided by an (implanted) sensor measuring respiration-related parameters. These may for example comprise a respiratory frequency, depth, volume, and/or data on whether, how often, and/or when apnea and/or Cheyne-Stokes respiration occurs.

In another example, the physiological data may alternatively or additionally be provided by an accelerometer collecting motion-related parameters and might, e.g., indicate falling of the patient and/or other forms of motion.

In examples, wherein the predetermined discovery signal comprises physiological data, the physiological data comprised in the discovery signal may be updated, e.g., every 1 h, 2 h, 3 h, 4 h, 6 h, 8 h, 12 h, 24 h, every second day, every week, every month, every year, etc. The content of the predetermined discovery signal may not be updated in response to a received trigger but independently from it such that the regularly or irregularly updated predetermined discovery signal is sent as it is upon receiving a trigger—in effect reporting the prevailing status of a condition or metric of interest.

According to a further aspect of the invention, a medical device for scanning its surrounding for discovery signal transmitted by implants is provided. The medical device comprises means for sending a trigger requesting a predetermined discovery signal and means for receiving the predetermined discovery signal.

As a further preferred secure embodiment of this invention, the system may generally report device type and manufacturer information but demand that direct physical contact with the patient occur before any additional patient-specific information is relayed from the implant. In effect, the system could identify what devices are in the “near neighborhood” using a non-contact approach but need to place the surveying/triggering device into direct physical contact with the patient (e.g., pressing it against the patient's chest) to collect information such as a metric pertaining to the physiologic status and wellbeing of the patient.

In general, this aspect and the exemplary embodiments in the following bear the advantages described herein in reference to the respective counterpart: the implant transmitting the discovery signal. The medical device capable of receiving one or more discovery signals from one or more implants may provide an opportunity to identify the presence of one or more implants in a patient's body, the device type and manufacturer quickly and efficiently. This may significantly reduce the time needed to check whether there are implants within the patient's body and to further identify the implants by providing a universal identification scheme. Said reduction of time may sideline many of the traditional needs for searching patient records, subjecting the patient to fluoroscopic imaging, and/or enforcing trial and error procedures using a potential multitude of manufacturer-specific programmers. The invention may be especially beneficial for patients in incapacitated conditions but may also offer benefits in cases where a conscious patient might not be aware of their specific implant type.

In other examples, the medical device may receive a discovery signal without sending a trigger. An according implant may, e.g., transmit a discovery signal periodically and/or when a certain event occurs. The medical device of such example may, e.g., be receptive to such discovery signals at least at certain, e.g. periodic, times.

The medical device may, e.g., comprise separate means configured to transmit the trigger and to receive the discovery signal or have one combined means for transmitting the trigger and receiving the discovery signal. The means for transmitting and receiving may be based for example on wireless communication methods like, e.g., Bluetooth low energy.

The trigger is configured to request a predetermined discovery signal to be transmitted by any device receiving the trigger, e.g., one or more implants of a patient, may be any signaling which is capable of evoking the transmission of said discovery signal. Such trigger may only evoke the transmission of a discovery signal when the trigger is received by an according implant. Therefore, the transmission of said discovery signal evidently indicates the presence of the implant transmitting the discovery signal and the discovery signal serves the purpose of allowing the implant to be discovered and/or to be identified.

The discovery signal may be characterized as described herein, e.g., in reference to any embodiment of a respective implant capable of transmitting said discovery signal. The medical device may, e.g., receive one or more discovery signals from one or more implants. For example, the medical device may process the incoming discovery signals and provide for example a list or overview based on the received discovery signals. The list may, e.g., list the respective implants that transmitted the discovery implants by implant type, implant manufacturer, and patient name or by any additional and/or other labels. The medical device may further comprise a user interface, e.g., a screen, for providing said list or overview.

The medical device may, e.g., be a handheld and/or mobile device and may be placed near the patient and/or on the patient's body, e.g., near to the expected position of any potential implants, e.g., on the chest of the patient. The medical device may, e.g., be a mobile phone with a corresponding application supporting the functions described herein. The medical device may further be operably connected to an electronic health record/data base comprising, e.g., electronic patient files.

In an exemplary embodiment, the trigger may comprise no instruction for determining the content of the requested discovery signal and the content of the discovery signal may be independent of, e.g., predetermined before receiving, the trigger.

The trigger does not comprise an instruction determining the content of the discovery signal but merely requests that a discovery signal is being sent by any implant receiving the trigger combined with the discovery signal being predetermined before receiving the trigger poses a rigid protocol. This may increase safety and reliability of sensitive patient data communication.

For example, this communication protocol may function without establishing and sustaining a connection in between the transmitter of the trigger and the implant receiving the trigger which may save energy and increase safety further. In other words, without needing a link that would be maintained only subject to a series of timeout mechanisms wherein regular data relay was required between an implant and a surveying device.

The trigger may for example simply comprise a request independent from, e.g., a device type and manufacturer of implants potentially receiving the trigger to send a discovery signal of any form, as the form and content of the discovery signal is being determined by the implant and not by the medical device transmitting the trigger.

The discovery signal is predetermined in such that its form and content are independent of the received trigger. In the underlying mechanism receiving the trigger directly evokes the transmission of the predetermined discovery signal. Therefore, the medical device may be perceptive to receiving a discovery signal of any form and/or content that may be unknown to the medical device before receiving it. The medical device may be configured to receive and recognize one or more discovery signals as such. When more than one discovery signal is received by the medical device, these may arrive at the same time or short after one another. The medical device may therefore be configured to receive and differentiate between such pluralities of incoming discovery signals.

For example, the trigger may be undirected and comprise no implant-specific information, the discovery signal may comprise at least an implant identification and/or a patient identification and/or the medical device may comprise means for decoding the implant identification and the patient identification and/or additional patient identification information, preferably in an encrypted manner.

The capability of the medical device to transmit undirected and non-implant-specific information within the trigger allows it to address any potential implant, this may, e.g., include implants that were developed after the medical implant and that could therefore not be known to the medical device and/or stored in a implant-specific sequence of an implant-specific trigger. This allows an open and unbiased scan for implants in the patient which may even be compatible with a growing plurality of (new) implants on the market. E.g., this prevents the case that the health professional assumes that the patient has no implant even though they do.

The discovery signal comprising at least an implant identification and/or a patient identification provides the opportunity to any receiver of a plurality of such discovery signals to differentiate between the implants of different patients and have an overview on which patient has which implants, e.g., in situations when patients share a room, etc. The medical device may, e.g., provide an overview sorted by patient such that the attending health professional operating the medical device gets an overview of the patient at hand immediately which reduces potential error sources. E.g., this prevents cases in which an implant of a second patient in the room next door may be detected by the medical device and the health professional might try to access and/or find said device within the body of a first patient without success, which might waste time and reduce treatment success chances.

The trigger signal not comprising implant-specific information relates to a general trigger comprising an anonymous request, instructing any device receiving the trigger to send a discovery signal to be then detected by the transmitter of the trigger or a second associated device. The trigger signal may be sent, e.g., based on user input. For example, the attending health professional may operate the medical device via, e.g., a user interface, a touch screen, buttons, levers, etc. to instruct the medical device to send one or more triggers. When more than one trigger is sent, for example, a succession of multiple triggers may be sent one after another to increase the chances that every implant of interest receives a trigger and transmits an associated discovery signal to be received by the medical device. The succession of one, two, three, four, five, ten, 15, 20, 25, 30, more, or any other number of triggers may be sent in a series separated by regular or irregular time intervals.

The discovery signal may comprise any information described herein, e.g., in reference to an embodiment of a respective implant for transmitting said discovery signal.

In an example, the medical device may further be configured to operate in a default state. It may send the trigger, based at least in part on a user input; transition into a scan state for a predetermined time after sending the trigger, wherein the medical device is configured to, in the scan state, scan for and receive the discovery signal transmitted by one or more implants. Further, it may transition into the default state after the lapse of the predetermined period.

This automated workflow may improve the interaction between the medical device and any number of implants receiving the trigger and transmitting a discovery signal to be detected by the medical device. In detail, this may ensure that the medical device is perceptive, e.g., by being in the scan state at the time when one or more discovery signals may arrive at the medical device. This may improve the reliability of the medical device in detecting all incoming discovery signals evoked by the respective trigger.

The medical device may, for example, send one trigger or a succession of multiple triggers and transition into a scan state before, during or after sending the trigger. For example, the medical device may comprise different means for transmitting the trigger and receiving discovery signals. In this example, the means for receiving may transition into the scan state even before the first trigger is sent. In examples where the same means may be provided for sending the trigger(s) and receiving the discovery signals(s), the means for sending and receiving may, e.g., transition into the scan state after the last trigger of the succession of trigger signals. The medical may remain within the scan state for a predetermined time for, e.g., 100 ms, 200 ms, 300 ms, 400 ms, 500 ms, 600 ms, 700 ms, 800 ms, 900 ms, 1 s, 2 s, 3 s, 4 s, 5 s, 6 s, 7 s, 8 s, 9 s, 10 s, 15 s, 20 s, 25 s, 30 s, shorter, longer, or any other time. The predetermined period may be set to a value that is long enough to ensure that the medical device does not leave the scan mode before an incoming discovery signal is received and, at the same time, short enough to not unnecessarily scan the surrounding at times when no triggered discovery signals may be expected. E.g., this prevents detecting discovery signals evoked by a second medical device that sent a trigger.

In addition, the scan and discovery approach may be designed such that any single implant (medical device) responds with a slight delay relative to the incoming trigger. This delay may be pseudo-random as facilitated by the trigger including a “seed” value that in combination with some implant-specific characteristic such as a serial number determines how long after the trigger a given implant waits to reply. The different delays associated with the in-range implants, in turn, spread out the implant responses over time (i.e., time multiplexing) to help avoid conditions where replies from any single implant would contaminate (i.e., jam or corrupt) the ability of the scanning device (medical device for scanning) to “hear” responses from other devices. In effect, this approach helps avoid having all in-range implants “speak” their responses at the same time—offsetting the challenges associated in understanding any single response.

In an exemplary embodiment, the medical device may further be configured to receive a plurality of discovery signals, wherein each discovery signal preferably has a different content and/or different properties.

The medical device may for example be configured to receive, recognize, and process multiple independent discovery signals. This may, e.g., comprise recognizing the common origin of the independent discovery signals, e.g., by comparison of the content of the independent discovery signals. In one example, they may all comprise an implant identification number allowing the device to group the as-received information besides the implant identification number, e.g., for displaying the information in an overview via the user interface of the medical device.

The properties of the independent discovery signals may vary, e.g., in terms of the associated privacy policies, decoding schemes, etc. The medical device may be configured to receive, recognize, and process the independent discovery signals together or separately.

According to a further aspect of the invention, a system comprising one or more implants as and a medical device as described herein.

Such system may bear all the advantages described herein in reference to the components of the system as well as synergistic effects between implants and the medical device. The mutual adjustment may improve and/or accelerate the implant identification workflow substantially.

The system may, e.g., comprise more than one medical device and more than one implant. For example, a system may also comprise only one implant and one medical device or one implant and more than one medical device.

According to another aspect, the invention provides a method to be carried out by an implant as described herein. The method comprises receiving a trigger requesting a predetermined discovery signal and transmitting the predetermined discovery signal.

Any other functionality described herein in reference to the implant, the trigger, and/or the discovery signal may be implemented as a respective step of a method.

For example, the method may further comprise transferring into a connection mode, wherein the implant is configured to establish a connection with an implant-specific programmer and establishing a connection with the implant-specific programmer. The attending health professional may directly know which of the available implant-specific programmers to use after identifying the respective implant as described herein.

In an exemplary scenario, the attending health professional may operate a medical device to send a trigger to one or more implants within a patient's body, e.g., in preparation of an emergency surgery where it is essential to save time. The implant(s) may each send a discovery signal such that the attending health professional may find and identify them. At this point, they may know, e.g., the respective device type(s) and manufacturer(s). Based on this knowledge, they may in a further step, use another device, namely for example an implant-specific and/or manufacturer-specific programmer, establish a connection with at least one of the detected implants to, e.g., transfer it into a mode that is compatible with the planned surgery and perform the respective surgery thereafter in a safe way.

According to another aspect, the invention provides a method to be carried out by a medical device for scanning its surrounding for discovery signals transmitted by implants as described herein. The method comprises sending a trigger requesting a predetermined discovery signal and receiving the predetermined discovery signal.

Any other functionality described herein in reference to the medical device, the trigger, and/or the discovery signal may be implemented as a respective step of a method.

The method may for example further comprise identifying an implant based on the received discovery signal and, at least in part based on the identifying, using an implant-specific programmer to establish a connection with the implant.

In an exemplary scenario, the attending health professional may operate the medical device by instructing it to send a trigger to one or more implants within a patient's body, e.g., in preparation of an emergency surgery where it is essential to save time. The implant(s) may each send a discovery signal. These may be received, recognized and/or processed by the medical device. The medical device may, e.g., output relevant information from the received discovery signal(s) via a user interface to make the information available to the attending health professional. Thus, the attending health professional may find and identify all relevant implants to be considered for their further proceeding. At this point, they may know, e.g., the respective device type(s) and manufacturer(s). Based on this knowledge, they may in a further step, use another device, namely for example an implant-specific and/or manufacturer-specific programmer, establish a connection with at least one of the detected implants to, e.g., transfer it into a mode that is compatible with the planned surgery and perform the respective surgery thereafter in a safe way.

Generally, the steps of the methods described herein in reference to the implant and the medical device, respectively, may combined in one method and further all steps of such methods may be implemented as instructions of a computer program.

Further, all functionalities described herein in reference to any implant, medical device and system embodiments may also be implemented as respective steps of a method and/or instructions of a computer program.

The method may for example further comprise using a second medical device for establishing a connection with the implant which is specific to the implant.

This may pose a time-efficient, safe, and reliable way of programming and/or accessing the implant and may involve existing implant-specific devices, e.g., implant-specific programmers, into the method.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 Schematic representation of a system comprising a medical device and an implant in a patient's body.

FIG. 2 Schematic representation of a discovery of a plurality of implants by two separate medical devices.

FIG. 3 Schematic representation of a workflow involving a medical device and an implant.

### DETAILED DESCRIPTION

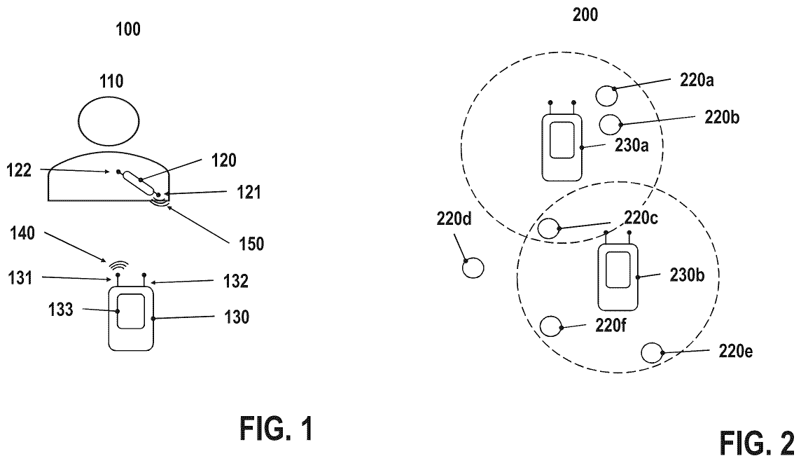

FIG. 1 shows a schematic representation of an exemplary system 100 comprising a medical device 130 and an implant 120 of a patient 110 exchanging signalling 140, 150.

The medical device 130 comprises a user interface 133, means for transmitting 131 (e.g., a trigger 140), and means for receiving 132 (e.g., a discovery signal 150). The implant 120 of FIG. 1 comprises means for transmitting 121 (e.g., a discovery signal 150), and means for receiving 122 (e.g., a trigger 140). The implant 120 is further implanted into a patient's 100 body but may, e.g., also be a partly implantable device. The patient 110 may optionally have further implants which are not shown in FIG. 1 but that might also be detected by the medical device 130.

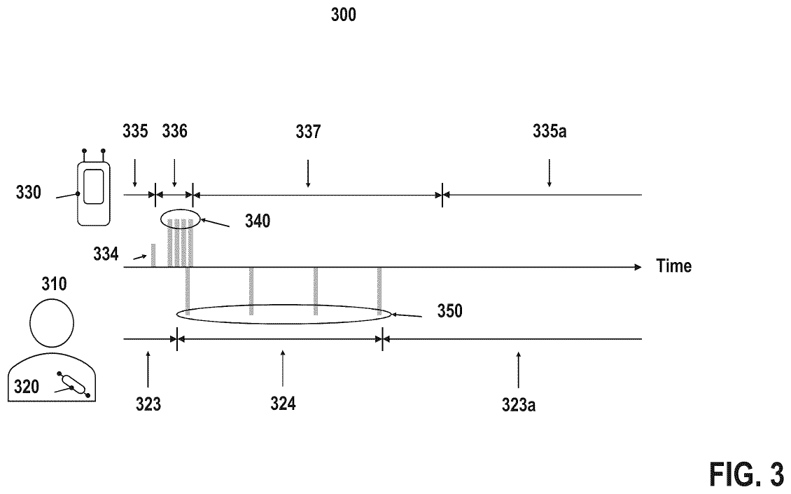

The medical device 130 of FIG. 3 transmits a trigger 140 by its means for transmitting 131. This may be induced, e.g., by user input by an attending health professional operating the medical device 130. The as-transmitted trigger 140 is undirected and may be received by any suitable receiver in the surrounding of the medical device 130. The trigger 140 of the exemplary system of FIG. 1 is received by means for receiving 122 a trigger 140 of the implant 120 which, in the example of FIG. 1, is the only suitable receiver in the surrounding of the medical device 130.

In response, the implant 120 of FIG. 1 sends a predetermined discovery signal 150 via the implant's 120 means for transmitting 121 the discovery signal 150. Neither the transmission of the trigger 140 nor of the discovery signal 150 requires a lasting connection between any of the devices participating in the signal exchange and may be based on wireless communication techniques, e.g., Bluetooth low energy.

The discovery signal 150 in the exemplary system 100 of FIG. 1 is received by the means for receiving 132 of the medical device 130. Once received, the discovery signal 150 may be processed by the medical device 130 or, e.g., by a remote server (not shown) that may be provided with the information of the discovery signal 150. Such exemplary remote server might then provide the processed, e.g., decoded, information to the medical device 130. The exemplary medical device 130 of FIG. 1 comprises a user interface 133 for outputting the as-received information comprised in the discovery signal 150 which may be used to output at least a part of the information of the discovery signal 150 to a user, e.g., the attending health professional.

There might be more implants (not shown) in reach of the medical device 130 wherein the trigger 140 might evoke sending a respective discovery signal (not shown) in an analogous process. In the exemplary system 100 of FIG. 1, all means for sending 121, 131 and all means for receiving 122, 132 are antennas but other embodiments may comprise other means for sending and receiving 121, 122, 131, 132. E.g., other embodiments may also combine the respective means for sending 121, 131 and means for receiving 122, 132 in the medical device 130 and/or the implant 120, respectively. Further, in other embodiments, the trigger 140 and the discovery signal 150 may be transmitted via, e.g., an additional relay device or a patient device, for example a mobile phone.

FIG. 2 shows a schematic representation of a plurality of devices 200 comprising two medical devices 230a, 230b and six implants 220a, 220b, 220c, 220d, 220e, 220f. The dashed circles illustrate the area that may be reached by a trigger signal transmitted by the respective medical device 230a, 230b in the center of the respective circle. Generally, there may be implants either within the area that may be reached by a trigger signal transmitted by the respective medical device 230a, 230b or not within that area.

In the example of FIG. 2, implants 220a and 220b may be implants of the same patient while implants 220c, 220d, 220e, 220f are of different patients each. There may be further patients (not shown) within reach of the medical devices 230a, 230b without in-body implants who may therefore not affect the plurality of devices 200 displayed in FIG. 2. The medical device 230a in a position as illustrated in FIG. 2 detects the implants 220a, 220b, 220c via the workflow described herein, e.g., in reference to FIG. 1. The medical device 230b in a position as illustrated in FIG. 2 detects the implants 220c, 220e, 220f via the workflow described herein, e.g., in reference to FIG. 1. Implant 220d is not detected by either of the exemplary medical devices 230a, 230b shown in FIG. 2. The first medical device 230a may, e.g., provide a first attending health professional operating the first medical device 230a with the information that two implants 220a, 220b are detected for a first patient and one implant 220c is detected for a second patient. The second medical device 230b may, e.g., provide a second attending health professional operating the second medical device 230b with the information that one implant 220c is detected for the second patient, one implant 220e is detected for a third patient, and one implant 220f is detected for a fourth patient. These information may, e.g., be provided along with an according device type and a manufacturer name.

FIG. 3 shows a schematic representation of an exemplary workflow 300 involving a medical device 330, e.g., as described herein and an implant 320 of a patient 310, e.g., as described herein. The actions relating to the medical device 330 are shown above the time-axis and the actions relating to the implant 320 are shown below the time-axis. In the exemplary workflow of FIG. 3, the medical device is initially in a default state 335 and the implant 320 is in a default receiving state 323. At the beginning, the medical device 330 (relating to the actions indicated above the time axis) is in a default state 335. The implant 320 (relating to the actions indicated below the time axis) is in a default receiving state 323. Signals of positive amplitude (above the time axis) correspond to signaling 340 transmitted by the medical device 330 or user input 334 at the medical device 330. Signaling with negative amplitude (below the time axis) corresponds to signaling 350 transmitted by the implant 320.

An attending health professional may initiate the workflow 300 of FIG. 3 via user input 334 instructing the medical device 330 to transition from the default state 335 into a phase 336 during which, in the example of FIG. 3, a series of four triggers 340 is being transmitted by the medical device 330. The number of triggers 340 transmitted during the phase 336 may be any other number in other examples. After transmitting the triggers 340, the exemplary medical device 330 of FIG. 3 automatically transitions into a scan state 337 for a predetermined time after sending the triggers 340 during which the medical device 330 may receive discovery signals 350. After the predetermined time, the medical device 330 transitions back into a default state 335a.

The phases 323, 324, 323a may overlap temporally with each other in other examples and analogously, the phases 335, 336, 337, 335a may in other examples overlap temporally with each other.

In the example of FIG. 3, the implant 320, initially in the default receiving state 323, receives the second trigger 340 transmitted by the medical device 330 and, in response, transitions into a discovery state 324, wherein a series of discovery signals 350 is being transmitted, for a predetermined period after receiving the trigger 340. The number of transmitted discovery signals 350, the duration between subsequent discovery signals 350, and/or the duration of the medical device 330 being in the scan state 337 may be adjusted to one another such as to ensure that at least one of the discovery signals 350 is received by the medical device 330. In FIG. 3, at least one of the discovery signals 350, e.g., as described herein, is received by the medical device 330. At the end of the discovery state 324, the implant 320 transitions back into the default receiving state 323a.

After receiving at least one discovery signal 350 from the implant 320, the medical device 330 may, e.g., process the as-received discovery signal(s) 350 as described herein and/or provide the information to the user via a user interface of the medical device 330 itself to any other remote device (not shown).

The analogous workflow 300 may for example be implemented for other durations of the respective phases, other numbers of transmitted triggers 340 and discovery signals 350, more medical devices 330, and/or more implants 320, etc.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implant for monitoring of a physiological parameter associated with a health state of a patient, the implant comprising:
means for receiving a trigger requesting a predetermined discovery signal; and
means for transmitting the predetermined discovery signal.

2. The implant of claim 1, wherein the trigger does not comprise an instruction determining the content of the requested discovery signal; and/or the content of the discovery signal is predetermined before receiving the trigger, preferably comprising at least an implant identification and/or a patient identification, preferably in an encrypted manner.

3. The implant of claim 1, wherein the trigger is undirected and/or does not comprise implant-specific information.

4. The implant of claim 1, further configured to operate in a default receiving state; upon receiving the trigger, transition into a discovery state for a predetermined period, wherein, in the discovery state, the discovery signal is transmitted; and transition into the default receiving state after the predetermined period lapses.

5. The implant of claim 1, further configured to transmit a plurality of discovery signals, wherein each discovery signal preferably has a different content and/or different properties; and/or update the content of the discovery signal, preferably based at least in part on physiological data acquired by the implant.

6. A medical device for scanning its surrounding for discovery signals transmitted by implants, the medical device comprising means for sending a trigger requesting a predetermined discovery signal; and means for receiving the predetermined discovery signal.

7. The medical device of claim 6, wherein the trigger does not comprise an instruction determining the content of the requested discovery signal; and/or the content of the discovery signal is independent of the trigger.

8. The medical device of claim 6, wherein the trigger is undirected and does not comprise implant-specific information; the discovery signal comprises at least an implant identification and/or a patient identification, preferably in an encrypted manner.

9. The medical device of claim 6, further configured to:
operate in a default state;
send the trigger, based at least in part on a user input;
transition into a scan state for a predetermined period after sending the trigger, wherein the medical device is configured to, in the scan state, scan for and receive the discovery signal transmitted by one or more implants; and
leave the scan state after the lapse of the predetermined period.

10. The medical device of claim 6, further configured to receive a plurality of discovery signals, wherein each discovery signal preferably has a different content and/or different properties.

11. A system comprising one or more implants according to claim 1 and a medical device for scanning its surrounding for discovery signals transmitted by the one of more implants, the medical device comprising means for sending a trigger requesting a predetermined discovery signal; and means for receiving the predetermined discovery signal.

12. A method, carried out by an implant, the method comprising the following steps: receiving a trigger requesting a predetermined discovery signal; and transmitting the predetermined discovery signal.

13. A method comprising the following steps carried out by a medical device for scanning its surrounding for discovery signals transmitted by implants: sending a trigger requesting a predetermined discovery signal; and receiving the predetermined discovery signal.

14. The method of claim 13, further comprising identifying an implant based on the discovery signal; and establishing a connection with the implant, at least in part based on the identifying.

15. The method of claim 14, further comprising using a second medical device for establishing a connection with the implant which is specific to the implant.
